SEQUENCE LISTING (48 sequences)

SEQ ID NO: 1 - DNA, 17 bp, Artificial Sequence
  Synthetic DNA
cctacgggrbgcascag

SEQ ID NO: 2 - DNA, 20 bp, Artificial Sequence
  Synthetic DNA
ggactacnngggtatctaat

SEQ ID NO: 3 - DNA, 17 bp, Artificial Sequence
  Synthetic DNA
cctacgggrbgcascag

SEQ ID NO: 4 - DNA, 20 bp, Artificial Sequence
  Synthetic DNA
ggactacyvgggtatctaat

SEQ ID NO: 5 - DNA, 20 bp, Artificial Sequence
  Synthetic DNA
agagtttgatcctggctcag

SEQ ID NO: 6 - DNA, 19 bp, Artificial Sequence
  Synthetic DNA
ggttaccttgttacgactt

SEQ ID NO: 7 - DNA, 17 bp, Artificial Sequence
  Synthetic DNA
ctacgggnggcngcagt

SEQ ID NO: 8 - DNA, 19 bp, Artificial Sequence
  Synthetic DNA
ggactaccngggtntctaa

SEQ ID NO: 9 - DNA, 20 bp, Artificial Sequence
  Synthetic DNA
agagtttgatcctggctcag

SEQ ID NO: 10 - DNA, 21 bp, Artificial Sequence
  Synthetic DNA
acggctaccttgttacgactt

SEQ ID NO: 11 - DNA, 19 bp, Artificial Sequence
  Synthetic DNA
tacchraggaggaagccac

SEQ ID NO: 12 - DNA, 22 bp, Artificial Sequence
  Synthetic DNA
gttcttcctaatctctacgcat

SEQ ID NO: 13 - DNA, 17 bp, Artificial Sequence
  Synthetic DNA
acgctacttgaggagga

SEQ ID NO: 14 - DNA, 18 bp, Artificial Sequence
  Synthetic DNA
gagccgtagcctttcact

SEQ ID NO: 15 - DNA, 20 bp, Artificial Sequence
  Synthetic DNA
gawgaagtatytcggtatgt

SEQ ID NO: 16 - DNA, 17 bp, Artificial Sequence
  Synthetic DNA
ctacgcwccctttacac

SEQ ID NO: 17 - DNA, 21 bp, Artificial Sequence
  Synthetic DNA
ctcaacttgggtgctgcattt

SEQ ID NO: 18 - DNA, 21 bp, Artificial Sequence
  Synthetic DNA
attgtagtacgtgtgtagccc

SEQ ID NO: 19 - DNA, 20 bp, Artificial Sequence
  Synthetic DNA
ttccggttgatccygccgga

SEQ ID NO: 20 - DNA, 19 bp, Artificial Sequence
  Synthetic DNA
yccggcgttgamtccaatt

SEQ ID NO: 21 - DNA, 20 bp, Artificial Sequence
  Synthetic DNA
tcygkttgatccygscrgag

SEQ ID NO: 22 - DNA, 16 bp, Artificial Sequence
  Synthetic DNA
tgggtctcgctcgttg

SEQ ID NO: 23 - DNA, 20 bp, Artificial Sequence
  Synthetic DNA
attagatacccsbgtagtcc

SEQ ID NO: 24 - DNA, 16 bp, Artificial Sequence
  Synthetic DNA
gccatgcaccwcctct

SEQ ID NO: 25 - DNA, 17 bp, Artificial Sequence
  Synthetic DNA
gygcascagkcgmgaaw

SEQ ID NO: 26 - DNA, 20 bp, Artificial Sequence
  Synthetic DNA
ggactacvsgggtatctaat

SEQ ID NO: 27 - DNA, 19 bp, Artificial Sequence
  Synthetic DNA
tggttgatcctgccagagg

SEQ ID NO: 28 - DNA, 19 bp, Artificial Sequence
  Synthetic DNA
cggctggcaccggtcttgc

SEQ ID NO: 29 - DNA, 32 bp, Artificial Sequence
  Synthetic DNA
ggtggtgtmggattcacacartaygcwacagc

SEQ ID NO: 30 - DNA, 23 bp, Artificial Sequence
  Synthetic DNA
ttcattgcrtagttwggrtagtt

SEQ ID NO: 31 - DNA, 20 bp, Artificial Sequence
  Synthetic DNA
gcmatgcarathggwatgtc

SEQ ID NO: 32 - DNA, 21 bp, Artificial Sequence
  Synthetic DNA
tcatkgcrtagttdggrtagt

SEQ ID NO: 33 - DNA, 17 bp, Artificial Sequence
  Synthetic DNA
taagggctgggccaagt

SEQ ID NO: 34 - DNA, 20 bp, Artificial Sequence
  Synthetic DNA
cacctagtycgcaragttta

SEQ ID NO: 35 - DNA, 19 bp, Artificial Sequence
  Synthetic DNA
cgwagggaagctgttaagt

SEQ ID NO: 36 - DNA, 18 bp, Artificial Sequence
  Synthetic DNA
taccgtcgtccactcctt

SEQ ID NO: 37 - DNA, 18 bp, Artificial Sequence
  Synthetic DNA
atcgrtacgggttgtggg

SEQ ID NO: 38 - DNA, 21 bp, Artificial Sequence
  Synthetic DNA
cacctaacgcrcathgtttac

SEQ ID NO: 39 - DNA, 20 bp, Artificial Sequence
  Synthetic DNA
gtaaacgatrytcgctaggt

SEQ ID NO: 40 - DNA, 17 bp, Artificial Sequence
  Synthetic DNA
ggtccccacagwgtacc

SEQ ID NO: 41 - DNA, 24 bp, Artificial Sequence
  Synthetic DNA
atcagttttcacatggsgattgat

SEQ ID NO: 42 - DNA, 16 bp, Artificial Sequence
  Synthetic DNA
gggttaagcccgggta

SEQ ID NO: 43 - DNA, 24 bp, Artificial Sequence
  Synthetic DNA
ttagtttttcacatgaaatactaa

SEQ ID NO: 44 - DNA, 16 bp, Artificial Sequence
  Synthetic DNA
aagttaagctcgggat

SEQ ID NO: 45 - DNA, 19 bp, Artificial Sequence
  Synthetic DNA
gtagtcatatgcttgtctc

SEQ ID NO: 46 - DNA, 20 bp, Artificial Sequence
  Synthetic DNA
tccgcaggttcacctacgga

SEQ ID NO: 47 - DNA, 21 bp, Artificial Sequence
  Synthetic DNA
gcaagtctggtgccagcagcc

SEQ ID NO: 48 - DNA, 20 bp, Artificial Sequence
  Synthetic DNA
cttccgtcaattcctttaag

(19) United States
(12) Patent Application Publication
(10) Pub. No.: US 2009/0311677 A1
(43) Pub. Date: Dec. 17, 2009

(54) Identification of contaminating bacteria in industrial ethanol fermentations

(76) Inventors: Andrew Reeves, Chicago, IL (US); Richard E. Tobey, St. Charles, IL (US)

Correspondence Address: CARDINAL LAW GROUP, Suite 2000, 1603 Orrington Avenue, Evanston, IL, 60201, US

(21) Appl. No.: 12/157,978

(22) Filed: Jun. 13, 2008

(51) Int. Cl. C12Q 1/68; C07H 21/04

(52) U.S. Cl. 435/6; 536/24.33

(57) ABSTRACT

A method for determining the presence of a contaminating organism in a fermentation process includes initiating the fermentation process using a syngas and obtaining a first aliquot from the fermentation process. The method further includes subjecting said first aliquot to a polymerase chain reaction using at least one oligonucleotide primer capable of hybridizing to a target sequence of a genomic nucleic acid from a suspected contaminating organism, producing a first amplified product based on the polymerase chain reaction, separating the first amplified product based on size, and determining the presence of the suspected contaminating organism based on the first separated amplified product.

### CROSS-REFERENCE TO RELATED APPLICATIONS

Not Applicable

### STATEMENT REGARDING FEDERALLY SPONSORED RESEARCH

Not Applicable

### THE NAMES OF THE PARTIES TO A JOINT RESEARCH AGREEMENT

Not Applicable

### INCORPORATION-BY-REFERENCE OF MATERIAL ON DISC

Not Applicable

### BACKGROUND

Bacterial contamination of industrial fermentations is a costly problem in terms of production time lost, wasted starting materials, and increased labor and decontamination costs. Heavy contamination of fermentation processes can temporarily shut down industrial operations in order to fully rid the plant of the infecting organism(s). While bacterial contamination is a difficult issue to eliminate completely from any fermentation facility, there are ways to reduce the likelihood of contaminating large-scale fermentation vessels and in effect mitigate downstream clean up costs, if they should arise. By instituting a contamination control program covering everything from testing stored stock cultures, seed cultures used for scale up into larger vessels, and pilot plant cultures, lost time, material costs and reduced fermentation performance can be minimized.

There are many ways to determine whether a culture broth contains a contaminating organism, either bacterial or non-bacterial. A mixture of organisms may be observed directly using various types of microscopy or by using a variety of stains to distinguish the different cell types. Alternatively, the mixture may be identified by growing a culture sample on solid media to identify different colony types, or by using analytical methods to identify unexpected metabolites, or by a variety of molecular biology-based techniques. All of these methods have advantages and disadvantages depending on when the test is performed. For example, while microscopy is often helpful, it is not thorough or quantitative enough to detect very low levels of contamination at any stage of the fermentation process. Staining methods are often inconclusive and suffer from background problems. Plating for the identification of pure isolates is limited in its utility if the contaminant is not cultivatable or requires special nutrients or growth conditions. And analytical techniques are useful if you know a priori what metabolites to look for, but fall short when testing stored culture stocks or cultures with very low levels of contamination. To date, molecular biology-based methods are by far the most sensitive and definitive.

Consequently, there is need for a reliable and reproducible method for identifying and distinguishing contaminants in all stages of an industrial fermentation process.

### SUMMARY

A first embodiment of the invention is a method for determining the presence of a contaminating organism in a fermentation process. The method may comprise initiating the fermentation process using at least one syngas input; (for purposes of this invention syngas comprises at least one of CO or H₂ and CO₂, and most often a mixture of CO, H₂, and CO₂) obtaining a first sample from the fermentation process; subjecting said first sample to a polymerase chain reaction using at least one set of oligonucleotide primers capable of hybridizing to a target sequence of a genomic nucleic acid from a suspected contaminating organism; producing a first amplified product based on the polymerase chain reaction; separating the first amplified product based on size; and determining the presence of the suspected contaminating organism based on the first separated amplified product.

Embodiments of the method further comprise obtaining a second sample from the fermentation process; subjecting said second sample to a polymerase chain reaction using at least one set of oligonucleotide primers capable of hybridizing to a target sequence of a genomic nucleic acid from a suspected contaminating organism; producing a second amplified product based on the polymerase chain reaction; separating the second amplified product based on size; comparing the first separated amplified product and second separated amplified product; and determining a change in contamination of the fermentation process based on the comparison.

Embodiments of the method further comprise obtaining a control sample including a known concentration of at least a first contaminating agent; subjecting said control sample to a polymerase chain reaction using at least one set of oligonucleotide primers capable of hybridizing to a target sequence of said control sample; producing a control amplified product based on the polymerase chain reaction; separating the control amplified product based on size; and comparing the first separated amplified product and the control amplified product, wherein the determination of the presence of the suspected contaminating organism is additionally based on the comparison.

In embodiments of the method, the contaminating organism may be a microorganism selected from the group consisting of archaea, bacteria, and, phage. In further embodiments, the contaminating organism may be Clostridia or Methanogen.

In embodiments of the method, the first or second sample may be selected from the group consisting of glycerol stocks, lyophilized cultures, agar stabs, agar plated cultures, seed cultures, effluent cultures and biofilm cultures. In further embodiments, the first or second sample may be a fermentation aliquot selected at time point from a fermentation process. For purposes of taking the samples, the fermentation process is defined as all steps taken in the preparation of the microorganism for commercial scale use from inoculation of starter cultures from preserved stocks to large-scale commercial production of liquid products from syngas inputs. Specifically, this would include initial culture inoculation from frozen stocks or freeze-dried stocks, vegetative starter cultures, scaled-up starter and seed cultures, pilot-scale cultures and large-scale industrial fermentations in a variety of vessel types. Amplification products from these time points may be compared to determine a change in the presence, absence, or amount of the suspected contaminating organism in said sample.

Embodiments of the method further comprise before the separation by size, reacting the first amplified product with at least one restriction endonuclease to produce a set of shorter fragments of the first amplified product, wherein a pattern of said shorter fragments identifies said contaminating organism in said sample.

In embodiments of the method, the separation by size may be by gel electrophoresis.

In embodiments of the method, the target sequence of said genomic nucleic acid is a portion of a 16s ribosomal RNA or 16s ribosomal DNA.

In yet further embodiments of the method, additional sets of said oligonucleotide primers may be used, each set of oligonucleotide primers being specific for a target sequence from a different suspected contaminating organism.

In additional embodiments of the method, the oligonucleotide primers may be selected from the group consisting of SEQ ID Nos. 1-26 and 29-46.

Embodiments of the method further comprise, after the step of obtaining a first sample from the fermentation process, subjecting said first sample to at least one sample preparation step. A known amount of at least one known nucleic acid molecule may be added as an internal standard, wherein at least one known nucleic acid molecule is capable of hybridizing with the same set of oligonucleotide primers used to amplify a target sequence of a genomic nucleic acid from the suspected contaminating organism. After the PCR amplification and size separation steps, an amount of amplified internal standard product may be quantitated, wherein this amount may be used as a standard for quantitative determination of an amount of the first amplified product.

A second embodiment of the invention is a method for determining the presence of a contaminating organism in a fermentation process. The method may comprise initiating the fermentation process using at least one syngas input; obtaining a first sample from the fermentation process; subjecting the first sample to a molecular beacon assay using at least one oligonucleotide probe capable of hybridizing to a target sequence of a genomic nucleic acid from a suspected contaminating organism to produce a first visible signal; quantifying the first visible signal; and determining the presence of the suspected contaminating organism based on said first visible signal.

Embodiments of this second method may further comprise obtaining a control sample including a known concentration of at least a first contaminating agent; subjecting said control sample to a molecular beacon assay using at least one oligonucleotide probe capable of hybridizing to a target sequence of a genomic nucleic acid from said control sample to produce a visible control signal; quantifying the visible control signal; and comparing the first visible signal and said visible control signal, wherein the determination of the presence of the suspected contaminating organism is additionally based on the comparison.

Yet another embodiment of the invention is an oligonucleotide primer for use in a PCR assay for determining the presence, absence, or amount of a contaminating organism in a fermentation process, selected from the group consisting of: 5′-ATC AGT TTT CAC ATG GAG ATT GAT-3′ (SEQ ID No. 43); 5′-GGG TTA AGC CCG GGT A-3′ (SEQ ID No. 44); 5′-TTA GTT TTT CAC ATG AAA TAC TAA-3′ (SEQ ID No. 45); and 5′-AAG TTA AGC TCG GGA T-3′ (SEQ ID No. 46).

An additional embodiment of the invention is a kit for detecting the presence, absence, or amount of a contaminating organism in a syngas fermentation to produce liquid products, such as ethanol, comprising: at least one set of oligonucleotide primers, wherein the primers may be able to hybridize to a target sequence of a genomic nucleic acid of a suspected contaminating organism to produce an amplified product in a PCR assay; a control sequence; and a set of control oligonucleotide primers, wherein the control primers may be able to hybridize to the control sequence to produce an amplified product of a known length in a PCR assay. In embodiments of the kit, the oligonucleotide primers may be selected from the group consisting of SEQ ID Nos. 1-26 and 29-46. In further embodiments of the kit, the target sequence of the genomic nucleic acid may be a portion of a 16s ribosomal RNA or 16s ribosomal DNA.

An additional embodiment of the invention is a second kit for detecting the presence, absence, or amount of a contaminating organism in a syngas fermentation to produce liquid products, comprising: at least one oligonucleotide probe, wherein the probe may be able to hybridize to a target sequence of a genomic nucleic acid of a suspected contaminating organism to produce a visible signal; a control sequence; and a control oligonucleotide probe, wherein the control probe may be able to hybridize to the control sequence to produce a visible signal. In embodiments of this second kit, the oligonucleotide probe and control oligonucleotide probe may be molecular beacons.

### BRIEF DESCRIPTION OF THE DRAWINGS

Aspects, features, benefits and advantages of the embodiments herein will be apparent with regard to the following description, appended claims, and accompanying drawings, where:

FIG. 1 depicts an exemplary flow diagram of a method for determining the presence of a contaminating organism in a fermentation process in accordance with one aspect of the invention.

FIG. 2 depicts an exemplary kit for detecting the presence of a contaminating organism in a fermentation process in accordance with one aspect of the invention.

FIG. 3 depicts an exemplary kit for detecting the presence of a contaminating organism in a fermentation process in accordance with one aspect of the invention.

FIG. 4 illustrates an exemplary electrophoretic profile of rDNA amplification products from pure cultures and mixed populations using Clostridia and archaeal-based primers on an ethidium bromide stained 1% agarose gel. Reactions contained Clostridium carboxidovorans cells and the following 16s ribosomal RNA domain-specific amplification primers: Eubacterial specific, SEQ ID NOs. 7, 8 (lane 2); Clostridium group I, SEQ ID NOs. 11, 12 (lane 3); Clostridium group II, SEQ ID NOs. 13, 14 (lane 4); Clostridium group III, SEQ ID NOs. 15, 16 (lane 5); Clostridium all groups, SEQ ID NOs. 17, 18 (lane 6); Archaeal, SEQ ID NOs. 19, 20 (lane 7). Lane 8 contains Methanococcales jannaschii genomic DNA as an archaeal control and lane 9 contains C carboxidovorans cells and M jannaschii genomic DNA. Lane 1 is a 1 Kb mass ladder.

FIG. 5 illustrates an exemplary electrophoretic profile of rDNA amplification products from pure cultures and mixed populations using Clostridia and archaeal-based primers on an ethidium bromide stained 1% agarose gel. Reactions contained Clostridium ragsdalei cells and the following 16s ribosomal RNA domain-specific amplification primers: Eubacterial specific, SEQ ID NOs. 7, 8 (lane 1); Clostridium group I, SEQ ID NOs. 11, 12 (lane 2); Clostridium group II, SEQ ID NOs. 13, 14 (lane 3); Clostridium group III, SEQ ID NOs. 15, 16 (lane 4); Clostridium all groups, SEQ ID NOs. 17, 18 (lane 5); Archaeal, SEQ ID NOs. 19, 20 (lane 6). Lane 7 contains Methanococcales jannaschii genomic DNA as an archaeal control and lane 8 contains C ragsdalei cells and M jannaschii genomic DNA.

FIG. 6 illustrates an exemplary electrophoretic profile of rDNA amplification products from pure cultures of Clostridia using clostridia-based primers on an ethidium bromide stained 1% agarose gel. Reactions contained: Clostridium carboxidovorans cells and 16s ribosomal RNA domain-specific amplification primers specific for C carboxidovorans, SEQ ID NOs. 43, 44 (lanes 2, 3, 4 are three different annealing temperatures, 46° C., 50° C. and 58° C., respectively); Clostridium ragsdalei cells and 16s ribosomal RNA domain-specific amplification primers specific for C ragsdalei, SEQ ID NOs. 45, 46 (lanes 5, 6, 7 are three different annealing temperatures, 46° C., 50° C. and 58° C., respectively). Lane 1 is a 1 Kb mass ladder; arrow points to location of 420 bp amplification product.

FIG. 7 illustrates an exemplary electrophoretic profile of rDNA amplification products from pure cultures of Clostridia using clostridia-based 16s ribosomal RNA domain-specific amplification primers on an ethidium bromide stained 1% agarose gel. Reactions contained: Clostridium carboxidovorans cells and C carboxidovorans specific primers, SEQ ID NOs. 43, 44 (lanes 2, 4 at annealing temperatures 46° C. and 50° C., respectively); Clostridium carboxidovorans cells and C ragsdalei specific primers, SEQ ID NOs. 45, 46 (lanes 3, 5 at annealing temperatures 46° C. and 50° C., respectively); Clostridium ragsdalei cells and C ragsdalei specific primers, SEQ ID NOs. 45, 46 (lanes 6, 8 at annealing temperatures 50° C. and 58° C., respectively); Clostridium ragsdalei cells and C carboxidovorans specific primers, SEQ ID NOs. 43, 44 (lanes 7, 9 at annealing temperatures 50° C. and 58° C., respectively). Lanes 1 and 10 are a 1 Kb mass ladder; arrow points to location of 420 bp amplification product.

### DETAILED DESCRIPTION

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope in the present disclosure which will be limited only by the appended claims. Various scientific articles, patents and other publications are referred to throughout the specification. Each of these publications is incorporated by reference herein in its entirety.

It must also be noted that as used herein and in the appended claims, the singular forms “a”, “an”, and “the” include the plural reference unless the context clearly dictates otherwise. Thus, for example, reference to an “organism” is a reference to one or more organisms and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

### Definitions

“Optional” or “optionally” means that the subsequently described event or circumstance may or may not occur, and that the description includes instances where the event occurs and instances where it does not. In addition, the word “comprising” as used herein means “including, but not limited to”. Throughout the specification of the application, various terms are used such as “primary”, “secondary”, “first”, “second”, and the like. These terms are words of convenience in order to distinguish between different elements, and such terms are not intended to be limiting as to how the different elements may be utilized.

As used herein, “isolated” means altered or removed from the natural state through human intervention. For example, a ribosomal RNA naturally present in a living animal is not “isolated,” but a synthetic ribosomal RNA primer, or a ribosomal RNA fragment partially or completely separated from the coexisting materials of its natural state is “isolated.” An isolated ribosomal RNA primer or fragment can exist in substantially purified form, or can exist in a non-native environment such as, for example, a cell into which the primer or fragment has been delivered. Thus, the term “isolated nucleic acid fragment” will refer to a polymer of RNA or DNA that is single- or double-stranded, optionally containing synthetic, non-natural or altered nucleotide bases. A nucleic acid fragment in the form of a polymer of DNA may be comprised of one or more segments of cDNA, genomic DNA or synthetic DNA.

As used herein, the term “organism” is meant to include microorganisms such as, for example, bacteria, archaea, and phage. In embodiments, the organism may be a unicellular microorganism, wherein the microorganism may be a prokaryote. Examples of bacterial phyla of prokaryotes which are useful in the method of the invention include, but are not limited to Acidobacteria, Actinobacteria, Aquificae, Bacteroidetes, Chlamydiae, Chlorobi, Chloroflexi, Chrysiogenetes, Cyanobacteria, Deferribacteres, Deinococcus-Thermus , Dictyoglomi, Fibrobacteres, Firmicutes, Fusobacteria, Gemmatimonadetes, Nitrospirae, Planctomycetes, Proteobacteria, Spirochaetes, Thermodesulfobacteria, Thermomicrobia, Thermotogae and Verrucomicrobia. In embodiments, a specific phylum of bacteria may be firmicutes, which includes the class Clostridia and genus clostridium. Examples of archaea phyla of prokaryotes which are useful in the method of the invention include, but are not limited to Crenarchaeota, Euryarchaeota, Korarchaeota, Nanoarchaeota and ARMAN. In embodiments, a specific phylum of archaea may be Euryarchaeota, which includes the methanogens. In alternate embodiments, the method may be used to detect unicellular eukaryotic contaminants such as, for example, yeast.

As used herein, the term “fermentation process” shall be taken to mean any process for the production of energy in a cell under aerobic or anaerobic conditions. The fermentation process may be a fermentation using microorganisms capable of converting synthesis gas (abbreviated syngas) to other chemicals with the term syngas input referring to the supply of syngas constituting any one or all of the components CO, H₂, or CO₂. Samples from a fermentation process may be taken at any stage, and may thus comprise preservation stocks (e.g., lyophilized cultures, agar stabs, or stocks stored in liquid nitrogen or at sub-freezing temperatures [−20° C. or −80° C.] such as glycerol stocks), samples from an active fermentation process (e.g., agar plated cultures, liquid starter cultures, effluent cultures or samples at various time points during a fermentation) or biofilm cultures. The fermentation process will typically produce liquid products. Most often the liquid product will be ethanol, n-butanol, hexanol, acetic acid, butyric acid, combinations thereof, or the like, depending on the syngas and culture selected. Those skilled in the art will appreciate that numerous combinations of syngas and culture can be selected as desired for generating a particular liquid product desired.

The letters “A”, “G”, “T” and “C”, when referred to in the context of nucleic acids, will mean the purine bases Adenine (C5H5N5), Guanine (C5H5N5O) and the pyrimidine bases Thymine (C5H6N2O2) and Cytosine (C4H5N3O), respectively. Further, several abbreviations may be used, which when referred to in the context of nucleic acids, will mean a modified base or a specific grouping of the bases: “I” will mean Inosine (C5H3N4O), “U” will mean Uracil (C4H3N2O2), “R” will mean a purine base (A, G), “Y” will mean a pyrimidine base (C, T), “K” will mean a keto base (G, T), “M” will mean a amino base (A, C), “S” will mean a base capable of three hydrogen bonds (G, C), “W” will mean a base capable of two hydrogen bonds (A, T), “B” will mean any base other than A (G, C, T), “D” will mean any base other than C (G, A, T), “H” will mean any base other than G (A, C, T), “V” will mean any base other than T (G, C, A), and “N” will mean any of the four bases G, C, A, T.

“Gene” refers to a nucleic acid fragment that expresses a specific protein, including regulatory sequences preceding (5′ non-coding sequences) and following (3′ non-coding sequences) the coding sequence.

The term “oligonucleotide” refers to primers, probes, oligomer fragments to be detected, labeled-replication blocking probes, oligomer controls, and shall be generic to polydeoxyribonucleotides (containing 2-deoxy-D-ribose), to polyribonucleotides (containing D-ribose) and to any polynucleotide which is an N glycoside of a purine or pyrimidine base (nucleotide), or modified purine or pyrimidine base. Also included in the definition of “oligonucleotide” are nucleic acid analogs (e.g., peptide nucleic acids) and those that have been structurally modified (e.g., phosphorothioate linkages). There is no intended distinction between the length of a “nucleic acid”, “polynucleotide” or an “oligonucleotide”.

The term “primer” refers to an oligonucleotide (synthetic or naturally occurring), which is capable of acting as a point of initiation of nucleic acid synthesis or replication along a complementary strand when placed under conditions in which synthesis of a complementary stand is catalyzed by a polymerase. Specifically, the term “primer” as used herein refers to a sequence comprising two or more deoxyribonucleotides or ribonucleotides, preferably more than three, and most preferably more than eight. Typically, short oligonucleotides of 12-20 bases may be used as amplification primers in a polymerase chain reaction (PCR) in order to obtain a particular nucleic acid fragment comprising the primers.

The term “probe” refers to an oligonucleotide (synthetic or occurring naturally), that is significantly complementary to a “fragment” and forms a duplexed structure by hybridization with at least one strand of the fragment. Typically, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques).

The term “complementary” is used to describe the relationship between nucleotide bases that are hybridizable to one another. For example, with respect to DNA, adenosine is complementary to thymine and cytosine is complementary to guanine. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Exemplary hybridization conditions are described, inter alia, in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization.

The term “amplification product” refers to portions of nucleic acid fragments that are produced during a primer directed amplification reaction. Typical methods of primer directed amplification include polymerase chain reaction (PCR), ligase chain reaction (LCR) or strand displacement amplification (SDA). If PCR methodology is selected, the replication composition would include for example, nucleotide triphosphates, two primers with appropriate sequences, DNA or RNA polymerase and proteins. Exemplary reagents and details describing procedures for their use in amplifying nucleic acids are provided in U.S. Pat. No. 4,683,202 (1987, Mullis, et al.) and U.S. Pat. No. 4,683,195 (1986, Mullis, et al.). If LCR methodology is selected, then the nucleic acid replication compositions would comprise, for example, a thermostable ligase, e.g., T. aquaticus ligase, two sets of adjacent oligonucleotides wherein one member of each set is complementary to each of the target strands, Tris HCl buffer, KCl, EDTA, NAD, dithiothreitol and salmon sperm DNA. Other methodologies that are appropriate can be found in, for example, Tabor et al., Proc. Acad. Sci. U.S.A., 82, 1074 1078 (1985) and Walker et al., Proc. Natl. Acad. Sci. U.S.A., 89, 392, (1992). Other methodologies may also be used.

Any appropriate recombinant DNA and molecular cloning techniques such as those described by Sambrook et al. (1989; supra) and by Ausubel, F. M. et al., Current Protocols in Molecular Biology, published by Greene Publishing Assoc. and Wiley-Interscience (1987), but including other techniques, can be used within the scope of these disclosures.

### PCR Assay Methods

Sequences of the present invention may be used as primers or to generate primers that may be used in primer directed nucleic acid amplification to detect the presence of a contaminating organism. A variety of primer directed nucleic acid amplification methods are known in the art including thermal cycling methods such as polymerase chain reaction (PCR) and ligase chain reaction (LCR) as well as isothermal methods and strand displacement amplification (SDA). In embodiments of the invention, primers used in PCR-type amplification techniques may have different sequences and are not complementary to each other. Depending on the desired test conditions, the sequences of the primers may be designed to provide for both efficient and faithful replication of the target nucleic acid. Methods of PCR primer design are described in several places, such as Rychlik, W. (1993) In White, B. A. (ed.), Methods in Molecular Biology, Vol. 15, pages 31-39, PCR Protocols: Current Methods and Applications. Humana Press, Inc., Totowa, N.J.

In embodiments, PCR may employ two oligonucleotide primers that bind to a selected nucleic acid template (e.g., DNA or RNA). Primers useful in the present invention include oligonucleotide primers capable of acting as a point of initiation of nucleic acid synthesis within or adjacent to a target sequence. A primer can be isolated from a restriction digest by conventional methods, or can be produced synthetically. Primers may be single-stranded for maximum efficiency in amplification, but a primer may also be double-stranded. Double-stranded primers are first denatured (e.g., treated with heat) to separate the strands before use in amplification. Primers can be designed to amplify a nucleotide sequence from a particular microbial species, or can be designed to amplify a sequence from more than one species. Primers that can be used to amplify a nucleotide sequence from more than one species are referred to herein as “universal primers.”

PCR assays may employ template nucleic acids such as DNA or RNA, including messenger RNA (mRNA) and ribosomal RNA (rRNA). The template nucleic acid need not be purified; it can be a minor fraction of a complex mixture, such as the DNA from a contaminating organism contained in a fermentation culture. Thus, the template may exist in whole cells from a resuspended culture broth. Alternately, template DNA or RNA may be extracted from a sample using a sample preparation step.

Further, the PCR amplification product may be digested with a restriction endonuclease, or set of restriction endonucleases, to provide a unique pattern (restriction fragment length polymorphism or RFLP) on an electrophoresis gel. Such a pattern may allow for more specific identification of a contaminating organism in a preservation stock or fermentation sample.

Within the context of the present invention, primers may be designed to regions of the 16S rDNA profile, 17S rDNA profile, 18S rDNA profile, or genes which are either common to all microorganisms (heat shock proteins, HSP's) or are unique to specific classes of organisms (methyl coenzyme M reductase, MCR; archaea) and which will be diagnostic of contamination by particular bacterial or non-bacterial species. Further, the primers may be designed to recognize variable regions of 16S rDNA profile, 17S rDNA profile, 18S rDNA profile or genes which may allow more refined classification within a genus, species or subspecies.

### Quantitative PCR (QPCR) Chemistries

In embodiments of the present invention, quantitative PCR (QPCR) may be used to determine the level of contaminating nucleic acid and by extrapolation the level of a contaminating microorganism in a fermentation sample. The amount of a target nucleic acid in a sample may be estimated by comparison with standard curves constructed from amplifications of serial dilutions of known nucleic acid molecules.

In RT-PCR, the accumulation of an amplification product is measured continuously in both standard dilutions of a known nucleic acid molecule and samples containing unknown amounts of a target sequence. A standard curve may be constructed by correlating initial template concentration in the standard samples with the number of PCR cycles necessary to produce a specific threshold concentration of an amplification product. In the test samples, target PCR product accumulation is measured after the same number of cycles, which allows interpolation of target DNA concentration from the standard curve.

### Detection of Amplification Products

Once a PCR amplification product is generated, it may be detected by, for example, electrophoresis and/or hybridization. In certain embodiments, the detection may be performed by visual means. A visualization method may include staining of a gel with ethidium bromide and visualization of bands under UV light. Alternatively, if the amplification products are integrally labeled with radio- or fluorometrically-labeled nucleotides, the separated amplification products may be subjected to radioactive scintigraphy of the incorporated radiolabel or fluorescent detection. Hybridization may be by Southern blot analysis or by use of a binding partner. For example, following separation of amplification products, a labeled nucleic acid probe may be brought into contact with the amplified product. The probe may be conjugated to a chromophore or a radiolabel. In another embodiment, the probe may be conjugated to a binding partner, such as an antibody or biotin, or another binding partner carrying a detectable moiety. Both electrophoresis and southern blot hybridization between nucleic acid molecules is discussed in detail in, for example, Sambrook et al. (1989, supra).

Thus, if contamination is present in a fermentation sample, a complex is formed between the contaminating organism's nucleic acid and a set of oligonucleotide primers, allowing for amplification of a product of a unique size and/or sequence. The presence of an amplification product is indicative of the presence of a contaminating organism in the fermentation sample. The amplification product formed may be detected directly on an electrophoresis gel (polyacrylamide or agarose), on a membrane or from an autoradiograph using, for example, a PhosphorImager or a Densitometer.

In certain embodiments, once a contaminant has been identified, quantitative RT-PCR may be used to determine the amount of a contaminating organism in the fermentation process sample. The amount or intensity of an amplification product from an RT-PCR assay is indicative of the amount of a contaminating organism in the fermentation sample.

### Molecular Beacon and Sequence-Specific Probe Assay Methods

In embodiments of the present invention, “Molecular Beacons” may be single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore may be covalently linked to the end of one arm, while a quencher may be covalently linked to the end of the other arm. As such, a molecular beacon will not fluoresce when free in solution. However, when hybridized to a nucleic acid strand containing a target sequence, a molecular beacon may undergo a conformational change that enables the fluorophore to fluoresce brightly. In the absence of targets, the probe is dark, because the stem places the fluorophore so close to the nonfluorescent quencher that they transiently share electrons, eliminating the ability of the fluorophore to fluoresce. When the probe encounters a target molecule, it forms a probe-target hybrid that is longer and more stable than the stem hybrid. The rigidity and length of the probe-target hybrid precludes the simultaneous existence of the stem hybrid. Consequently, the molecular beacon undergoes a spontaneous conformational reorganization that forces the stem hybrid to dissociate and the fluorophore and the quencher to move away from each other, restoring fluorescence.

In another embodiment, “sequence-specific” linear fluorescent probes are used in the detection process. These probes contain both a fluorescent reporter dye attached at one end of the probe and a quencher attached at the other end. As long as these two molecules are maintained in close proximity, the fluorescence from the reporter is quenched and no fluorescence is detected at the reporter dye's emission wavelength. The probe is designed to anneal to one strand of the target sequence slightly downstream of one of the primers. As DNA polymerase extends that primer, it will encounter the 5′ end of the probe. Commercially available RT-PCR enzymes contain 5′-3′ nuclease activity, so when it encounters the probe it displaces and degrades the 5′ end, releasing free reporter dye into solution. Following the separation of the reporter dye and quencher, fluorescence can be detected from the reporter dye.

In embodiments of the present invention, a molecular beacon may be used to detect a target nucleic acid. For example, as with the PCR primers discussed above, molecular beacons may also be designed to target variable or nonvariable regions of 16S rDNA, 17S rDNA, 18S rDNA or genes which are either common to all microorganisms (heat shock proteins, HSP's) or are unique to specific classes of organisms (methyl coenzyme M reductase, MCR; archaea) and which will be diagnostic of contamination by particular bacterial or non-bacterial species. When the molecular beacon encounters the target molecule, the loop and a part of the stem may hybridize to the target causing a spontaneous conformational change that forces the stem apart. Thus, the quencher moves away from the fluorophore leading to the restoration of fluorescence. One major advantage of the stem-loop probes of the current invention is that they may recognize target nucleic acids with a higher specificity than the linear oligonucleotide probes. Properly designed molecular beacons may be able to discriminate between targets that differ by as little as a single nucleotide. The molecular beacons may be utilized in real-time monitoring of PCR to detect and quantify specific transcripts (i.e., signature transcripts for methanogens, etc.) which would indicate, even at a vanishingly small level (1 cell in a billion or greater), the presence of a contaminating organism.

### 16S, Ribosomal RNA as a Target

In certain circumstances, ribosomal RNA molecules are used to uniquely identify a wide range of contaminating organisms in a fermentation process. Each ribosome is composed of three distinct ribosomal RNA molecules and a variety of protein molecules. In bacteria and archaea, the medium sized ribosomal RNA molecule, i.e., the 16S ribosomal RNA, molecule is particularly useful for determination of an organism's identity (Woese, C. R. 1987. Bacterial Evolution. Microbiol. Rev. 51:221 271).

The nucleotide sequence of the 16S ribosomal RNA molecule has conserved regions that are present in most if not all bacteria and archaea, and variable regions that may be used to distinguish species and subspecies. Since a ribosomal RNA molecule is a direct gene product that results from transcription of a corresponding ribosomal RNA gene (rDNA), rDNA may be specifically and rapidly isolated from a particular microorganism or a mixture of microorganisms using appropriate DNA primers and a PCR assay to amplify the rDNA. In embodiments of the invention, the presence of the rDNA fragment, as observed by electrophoresis (agarose or polyacrylamide gel of the amplified rDNA PCR product), or the pattern of fragments resulting from cutting the PCR product with a set of restriction endonucleases may be used to identify the organism from which the rDNA was amplified.

Alternately, in situ hybridization techniques using molecular beacon technology may be used to specifically and rapidly detect rDNA from a particular microorganism or a mixture of microorganisms using appropriate DNA probes (beacons) and spectroscopy to detect a fluorescent signal. Molecular beacons can be synthesized that possess differently colored fluorophores, enabling assays to be carried out that simultaneously detect different organisms in the same reaction.

Ribosomal RNA probes or primers may therefore be used to rapidly identify different organisms from broad genera to individual species. As such, the methods of this invention provide a sensitive method for rapidly determining the presence, absence or amount of a contaminating organism in any culture stock or broth.

### Identification of Contaminants

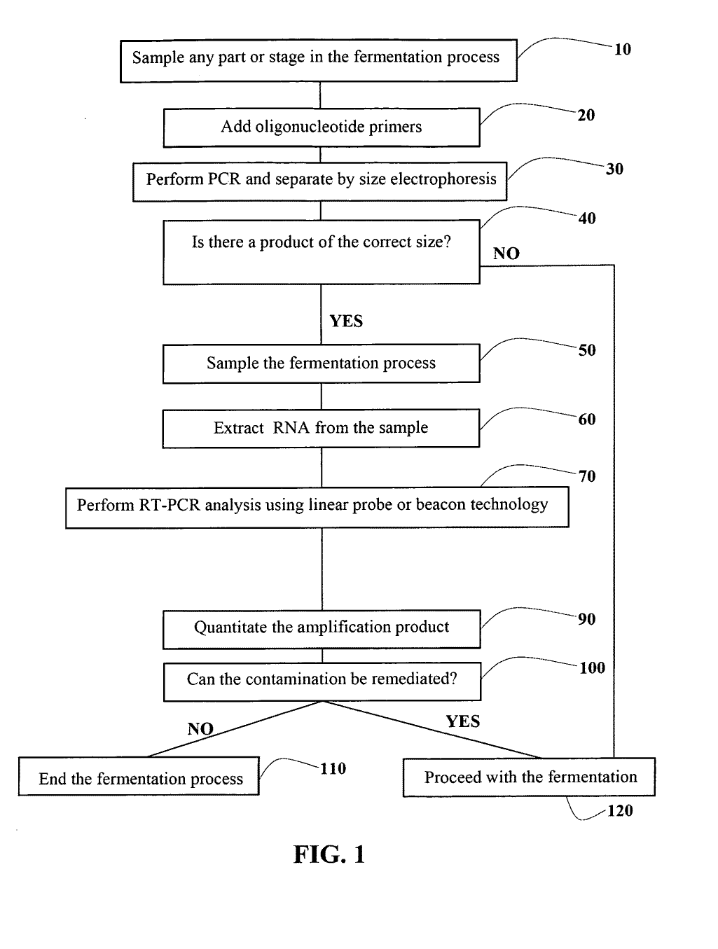

Embodiments of the present invention include methods of identification and quantification of a contaminating organism in a fermentation process. Referring to FIG. 1, an exemplary flow diagram for a method of determining the presence of a contaminating organism in a fermentation process is depicted. At step 10 in the flowchart, a sample of a fermentation process is obtained. The fermentation process may be any process for the production of energy in a cell under aerobic or anaerobic conditions. For example, the fermentation process may be a fermentation using microorganisms capable of converting a syngas input (any of CO, H₂, CO₂) to ethanol under anaerobic conditions. Such a fermentation process would be initiated using at least one syngas input. The sample obtained in step 10 may be taken at any stage in the fermentation process. For example, the sample may be taken from a preservation culture which is used to start a seed culture, which ultimately is used to start the fermentation process. Alternatively, the sample may be an aliquot from an active fermentation taken at any stage in fermentation. Thus, the sample may include stocks or samples which may be any of a: preservation stock (e.g., lyophilized cultures, agar stabs, or stocks which are stored in liquid nitrogen or at sub-freezing temperatures [−20° C. or −80° C.] such as glycerol stocks), samples from an active fermentation process (e.g., agar plated cultures, liquid starter cultures, effluent cultures or samples at various time points during a fermentation) or biofilm cultures.

An oligonucleotide primer or probe is added to the sample at step 20. The methods of identification involve the use of DNA primers or probes which target sequences of the 16S ribosomal RNA molecule, 17S ribosomal RNA molecule, 18S ribosomal RNA molecule or specific genes, and may be used in PCR or molecular beacon assays to routinely test a sample for the presence of many different organisms rapidly and accurately. For example, a sample from a fermentation process using Clostridium carbovidovorans cells could be contaminated with other strains of Clostridia, such as, for example Clostridium ragsdalei cells, or could be contaminated by an archaeal microorganism such as a methanogen. Several sets of primers for use in PCR analysis may be designed to a variable region of the 16S rDNA profile, such that each set of primers may be specific for Clostridium ragsdalei 16S rDNA or methanogen 16S rDNA. Alternately, probes which may act as molecular beacons may be designed to this same region of the 16S rDNA profile, such that each probe may be specific for Clostridium ragsdalei 16S rDNA or methanogen 16S rDNA. More than one set of primers or probes may be incorporated into a single sample assay.

A PCR analysis may then be performed, shown as step 30 in FIG. 1. If a PCR analysis is performed, the set of oligonucleotide primers added in step 20 may hybridize to a target region on the genomic nucleic acid from a suspected contaminating organism, and an amplification product may be generated. This amplification product may then be detected, using a size separation technique, such as by gel electrophoresis and staining with ethidium bromide. The appearance of a correctly sized product indicates the presence of the suspected contaminating organism in the fermentation sample. If more than one set of PCR primers was added to the sample in step 20, each set may be directed to a different suspected contaminating organism. Further, each set of PCR primers may be designed to generate amplification products of different sizes, allowing for easy identification of the various suspected contaminating organisms.

Thus, an embodiment of the invention is a method for determining the presence of a contaminating organism in a fermentation process. The method may comprise initiating the fermentation process using at least one syngas input; obtaining a first sample from the fermentation process; subjecting said first sample to a polymerase chain reaction using at least one set of oligonucleotide primers capable of hybridizing to a target sequence of a genomic nucleic acid from a suspected contaminating organism; producing a first amplified product based on the polymerase chain reaction; separating the first amplified product based on size; and determining the presence of the suspected contaminating organism based on the first separated amplified product.

Embodiments of the method further comprise obtaining a second sample from the fermentation process; subjecting said second sample to a polymerase chain reaction using at least one set of oligonucleotide primers capable of hybridizing to a target sequence of a genomic nucleic acid from a suspected contaminating organism; producing a second amplified product based on the polymerase chain reaction; separating the second amplified product based on size; comparing the first separated amplified product and second separated amplified product; and determining a change in contamination of the fermentation process based on the comparison.

Embodiments of the method further comprise obtaining a control sample including a known concentration of at least a first contaminating agent; subjecting said control sample to a polymerase chain reaction using at least one set of oligonucleotide primers capable of hybridizing to a target sequence of said control sample; producing a control amplified product based on the polymerase chain reaction; separating the control amplified product based on size; and comparing the first separated amplified product and the control amplified product, wherein the determination of the presence of the suspected contaminating organism is additionally based on the comparison.

In embodiments of the method, the contaminating organism may be a unicellular microorganism selected from the group consisting of archaea, bacteria, and phage. In further embodiments, the contaminating organism may be Clostridia and Methanogen. In embodiments of the method, the target sequence of said genomic nucleic acid may be a portion of a 16s ribosomal RNA or 16s ribosomal DNA.

In embodiments of the method, the first or second sample may be selected from the group consisting of glycerol stocks, lyophilized cultures, agar stabs, agar plated cultures, seed cultures, effluent cultures and biofilm cultures. The first or second sample may be a fermentation aliquot selected at time point from a fermentation process. Amplification products from these time points may be compared to determine a change in the presence, absence, or amount of the suspected contaminating organism in said sample. Embodiments of the method further comprise before the separation by size, reacting the first amplified product with at least one restriction endonuclease to produce a set of shorter fragments of the first amplified product, wherein a pattern of said shorter fragments identifies said contaminating organism in said sample. In embodiments of the method, the separation by size may be by gel electrophoresis.

In yet further embodiments of the method, additional sets of said oligonucleotide primers may be used, each set of oligonucleotide primers being specific for a target sequence from a different suspected contaminating organism.

In embodiments of the method, the oligonucleotide primers may be selected from the group consisting of SEQ ID Nos. 1-26 and 29-46 (see table 1).

If no amplification product from the PCR reaction is detected at step 40 of FIG. 1, then the fermentation may be determined to be free of contaminating organisms, and may progress as normal, as shown in step 120. Additional samples may be taken at later time points in the fermentation, or from a different preservation stock or culture. These samples may be tested as in steps 10 through 30 to ensure that a new contaminant has not been introduced to the fermentation process, or that a very slight contamination was not missed in the first analysis.

If an amplification product from the PCR reaction is detected at step 40 of FIG. 1, then the fermentation may contain a contaminating organism. Quantification of the amount of a contaminant may be determined using a quantitative technique such as RT-PCR. As shown in step 50, a sample of the fermentation process is obtained. The RNA from this sample is extracted using at least one sample preparation step, as shown in step 60. The sample is extracted using appropriate extraction techniques. In one embodiment, the sample is extracted using minimally invasive techniques. Often, steps will be taken to reduce contamination resulting from the sample extraction. The extracted RNA from this step may be used in an RT-PCR assay, as shown in step 70. The RT-PCR assay will contain an internal standard which allows quantitative detection of the PCR amplified product. Amplified products from the contaminating organism and the control internal standard may be quantitated, as shown in step 90. The amount and type of contaminant in the fermentation sample may determine whether remediation would be useful to rescue the fermentation process, as shown by the query in step 100. If remediation is possible, such as by addition of organism specific toxins or growth inhibitors, then the fermentation process may be continued, as shown in step 120. If remediation is not possible at step 100, then the fermentation process may be ended, as in step 110.

Thus, another embodiment of the invention is a method for quantifying the amount of a contaminating organism in a fermentation process. The method may comprise obtaining a sample from a fermentation process and subjecting the sample to at least one sample preparation step. The preparation step may isolate a ribonucleic acid (RNA) from the cells of the sample. The RNA is then converted to complementary DNA (cDNA) using reverse transcriptase and purified. A set of oligonucleotide primers which may hybridize to a target sequence of the genomic nucleic acid from the contaminating organism and a known amount of at least one known nucleic acid molecule which acts as an internal standard are added to the prepared sample. The internal standard may be able to hybridize with the same set of oligonucleotide primers used to amplify the target sequence of the genomic nucleic acid from the contaminating organism. The sample may then be subjected to a QPCR assay to produce an amplified internal standard product and an amplified product from the contaminating organism. The presence or absence of the amplified product in the sample indicates the presence or absence of the contaminating organism in the fermentation process. Further, the amount of amplified product may be quantified by comparison to the internal standard amplified product. A standard curve may be used to aid in quantification, wherein the standard curve is generated using serial dilutions of the internal standard.

Frequent testing using the methods of this invention may reduce loss of fermentation processes to contamination. Further, the rapid and sensitive nature of the methods of this invention may help to provide early detection of contamination in a fermentation process, thus reducing costly losses in production time, wasted starting materials, and increased labor and decontamination costs.

Yet another embodiment of the invention is an oligonucleotide primer for use in a PCR assay for determining the presence, absence, or amount of a contaminating organism in a fermentation process, selected from the group consisting of SEQ ID Nos. 43-46:

| | |
|---|---|
| 5′-ATC AGT TTT CAC ATG G AG ATT | (SEQ ID No. 43) |
| GAT-3′; | |
| 5′-GGG TTA AGC CCG GGT A-3′; | (SEQ ID No. 44) |
| 5′-TTA GTT TTT CAC ATG AAA TAC | (SEQ ID No. 45) |
| TAA-3′; | |
| and | |
| 5′-AAG TTA AGC TCG GGA T-3′. | (SEQ ID No. 46) |

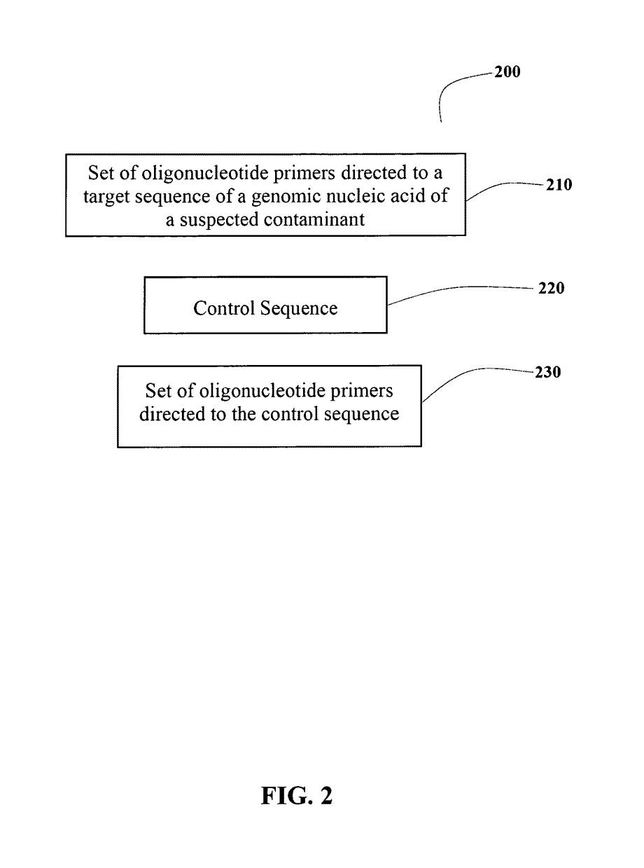

Embodiments of the invention include kits for detecting contamination in a fermentation process. The fermentation process may be any process for the production of energy in a cell under aerobic or anaerobic conditions. For example, the fermentation process may be a fermentation using microorganisms capable of converting syngas to ethanol. With reference to FIG. 2, an exemplary kit for detecting the presence of a contaminating organism in a fermentation process in accordance with one aspect of the invention is depicted. The kit (200) may contain a set of oligonucleotide primers directed to a target sequence of a genomic nucleic acid of a suspected contaminant (210). This set of primers may hybridize to the target sequence of the genomic nucleic acid and produce an amplified product in a polymerase chain reaction. The kit may also contain a control sequence (220) and a set of primers directed to the control sequence (230). The control sequence and control primers may be used to verify that the polymerase chain reaction is working efficiently. Further, the control sequence and control primers may be used as an internal standard in an RT-PCR assay to provide quantification of the amount of genomic nucleic acid of a suspected contaminant. RT-PCR assays on serial dilutions of the control sequence aid in the generation of a standard curve. Such a standard curve may then be used for comparison and quantification of the amount of a contaminating organism.

Thus, an additional embodiment of the invention is a kit for detecting the presence, absence, or amount of a contaminating organism in a fermentation process, comprising: at least one set of oligonucleotide primers, wherein the primers may be able to hybridize to a target sequence of a genomic nucleic acid of a suspected contaminating organism to produce an amplified product in a PCR assay; a control sequence; and a set of control oligonucleotide primers, wherein the control primers may be able to hybridize to the control sequence to produce an amplified product of a known length in a PCR assay. In embodiments of the kit, the oligonucleotide primers may be selected from the group consisting of SEQ ID Nos. 1-26 and 29-46. In further embodiments of the kit, the target sequence of the genomic nucleic acid may be a portion of a 16s ribosomal RNA or 16s ribosomal DNA.

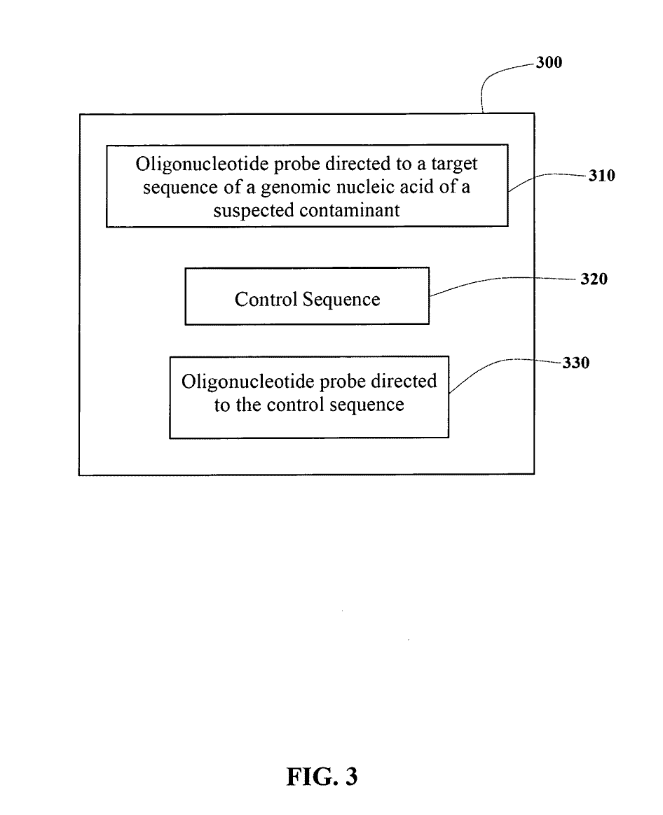

With reference to FIG. 3, an additional exemplary kit for detecting the presence of a contaminating organism in a fermentation process in accordance with one aspect of the invention is depicted. The kit (300) may contain oligonucleotide primers and probes directed to a target sequence of a genomic nucleic acid of a suspected contaminant (310). The primers and probes may hybridize to the target sequence of the genomic DNA and produce a visible signal in a QPCR assay. The kit may also contain a control sequence (320) and a probe directed to the control sequence (330). The control sequence and control probe may be used to verify that the QPCR assay is working efficiently. Further, the control sequence and control probe may be used as an internal standard to provide quantification of the amount of genomic nucleic acid of a suspected contaminant. Serial dilutions of the control sequence aid in the generation of a standard curve. Such a standard curve may then be used for comparison and quantification of the amount of a contaminating organism.

An additional embodiment of the invention is a second kit for detecting the presence, absence, or amount of a contaminating organism in a fermentation process, comprising: at least one molecular beacon or fluorescently-labeled probe, wherein the probe may be able to hybridize to a target sequence of a genomic nucleic acid of a suspected contaminating organism to produce a visible signal; a control sequence; and a control oligonucleotide probe, wherein the control probe may be able to hybridize to the control sequence to produce a visible signal. In embodiments of this second kit, the oligonucleotide probe and control oligonucleotide probe may be molecular beacons or fluorescently-labeled probes.

### EXAMPLES

### Determination of Contaminants in Fermentation Using Clostridia.

### General Methods

Group I Clostridia is an acetogenic bacterium which produces organic acids and solvents from synthesis gas via direct fermentation. Given the electron scavenging nature of these organisms and the low redox potential required to grow them successfully, the main predicted bacterial contaminants competing for electrons under syngas growth conditions would be the methanogens. Methanogens have been classified using the 16s ribosomal RNA phylogenetic classification system as archaeabacteria. The archaeabacteria (i.e., the methanogens, sulfate-reducing bacteria, halophiles, and extreme thermophiles among a few select others) differ from the eubacteria in a variety of cellular characteristics including their 16s ribosomal RNA sequences. The 16S ribosomal RNA has been shown to be a good indicator of bacterial evolution and divergence and may be used to classify thousands of species of organisms in a phylogentic classification scheme. Any desired classification scheme may be used, such as that proposed by Woese, 1989; supra. As such, probes or primers designed to target ribosomal RNA may provide an excellent means for rapid and sensitive identification of organisms from different genera to individual species.

Oligonucleotide PCR primers (table 1) were designed based on a variety of methanogen and clostridial 16S ribosomal RNA sequences to amplify specific DNA sequences that allow determination of the presence, absence or amount of a particular species or group such as a methanogen or clostridia in a fermentation sample. Comparison of the 16S ribosomal RNA sequences from the group I Clostridia and the Methanogens allows the synthesis of unique primers containing 17-20 nucleotides that, when used in a PCR reaction, amplify sequences which distinguish the Clostridia (Eubacteria) from the Methanogens (Archaeabacteria). This assay method is very sensitive and may be able to detect methanogen DNA or other contaminating DNA at the femtogram level.

The entire assay method, from taking a sample from the fermentation process to amplification and visualization in an electrophoretic gel may take as little as three hours. Thus, real-time analysis of a fermentation process is possible. All PCR reactions were carried out using an Eppendorf Mastercycler. Gel electrophoresis was carried out on 1% agarose gels, and visualization was done using a UVP BioImaging system.

### Verification and Validation of 16S-Based Ribosomal DNA Primers for PCR

An rDNA PCR amplification reaction was carried out using a set of PCR primers specific for Eubacterial (SEQ ID NOs. 7, 8), Clostridium group I (SEQ ID NOs. 11, 12), Clostridium group II (SEQ ID NOs. 13, 14), Clostridium group III (SEQ ID NOs. 15, 16), all groups of Clostridium (SEQ ID NOs. 17, 18), and Archaea (SEQ ID NOs. 19, 20). The test sample was either a culture of Clostridium carboxidovorans cells or Clostridium ragsdalei cells.

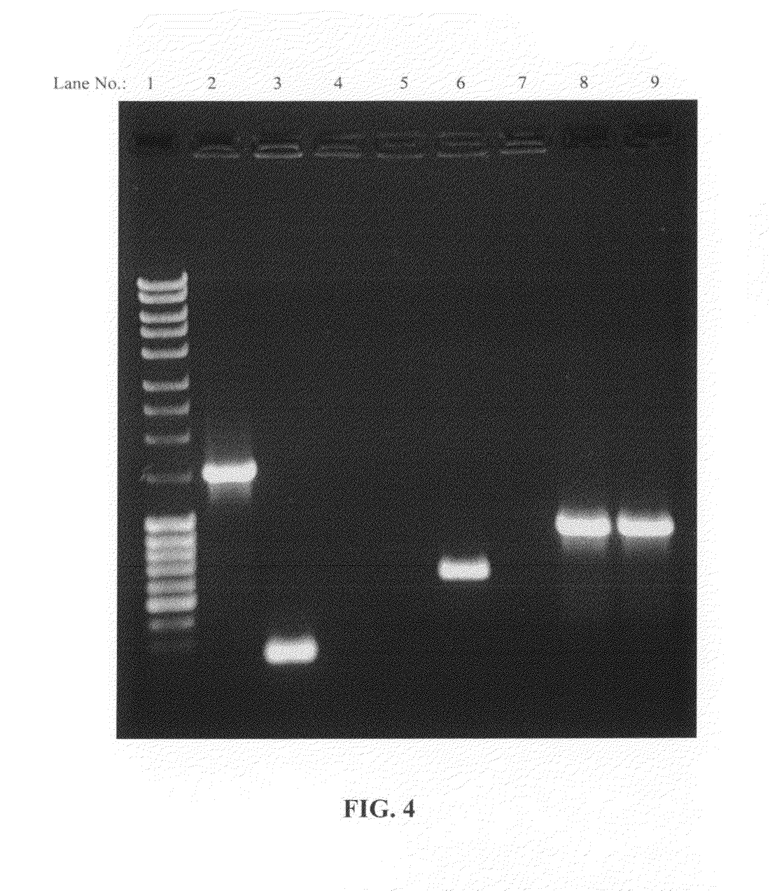

FIG. 4 is an ethidium bromide stained 1% agarose gel showing the electrophoretic profile for the rDNA amplification reaction products using the Clostridium carboxidovorans culture: (lane 2) Eubacterial specific primers; (lane 3) Clostridium group I specific primers; (lane 4) Clostridium group II specific primers; (lane 5) Clostridium group III specific primers; (lane 6) All Clostridium primers; (lane 7) Archaeal specific primers; (lane 8) Methanococcales jannaschii genomic DNA as an archaeal control; (lane 9) contains C carboxidovorans cells and M jannaschii genomic DNA; and (lane 1) 1 Kb mass ladder.

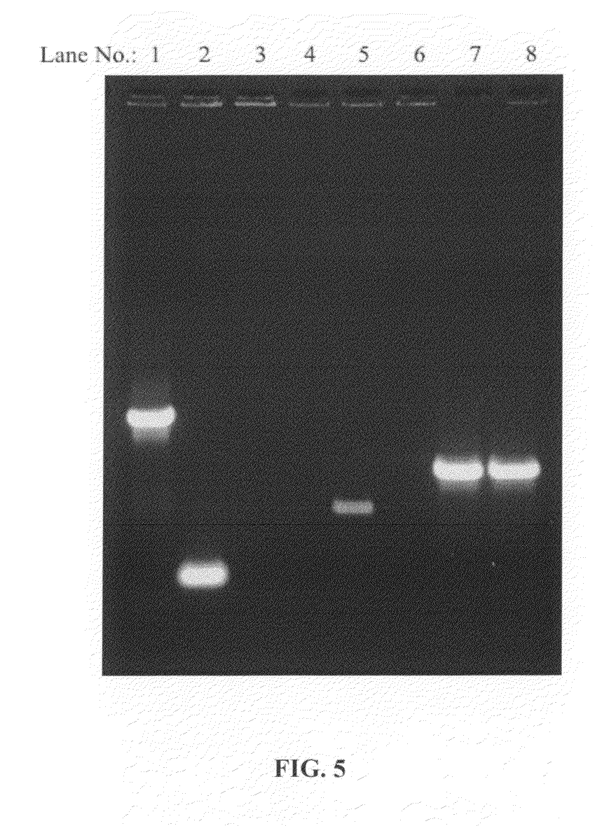

FIG. 5 is an ethidium bromide stained 1% agarose gel showing the electrophoretic profile for the rDNA amplification reaction products using the Clostridium ragsdalei culture: (lane 1) Eubacterial specific primers; (lane 2) Clostridium group I specific primers; (lane 3) Clostridium group II specific primers; (lane 4) Clostridium group III specific primers; (lane 5) All Clostridium primers; (lane 6) Archaeal specific primers; (lane 7) Methanococcales jannaschii genomic DNA as an archaeal control; and (lane 8) contains C carboxidovorans cells and M jannaschii genomic DNA.

TABLE I

| | | |
|---|---|---|
| SEQ | | |
| ID NO. | Sequence* | Target\hat{ } |

rDNA primers and their target organisms

| | | |
|---|---|---|
| 1 | 5′-CCT ACG GGR BGC ASC AG-3′ | All Bacteria and Eukarya |
| 2 | 5′-GGA CTA CNN GGG TAT CTA AT-3′ | |
| 3 | 5′-CCT ACG GGR BGC ASC AG-3′ | All Bacteria |
| 4 | 5′-GGA CTA CYV GGG TAT CTA AT-3′ | |
| 5 | 5′-AGA GTT TGA TCC TGG CTC AG-3′ | All Eubacteria |
| 6 | 5′-GGT TAC CTT GTT ACG ACT T-3′ | |
| 7 | 5′-CTA CGG GIG GCI GCA GT-3′ | All Eubacteria |
| 8 | 5′-GGA CTA CCI GGG TIT CTA A-3′ | |
| 9 | 5′-AGA GTT TGA TCC TGG CTC AG-3′ | All bacteria |
| 10 | 5′-ACG GCT ACC TTG TTA CGA CTT-3′ | |
| 11 | 5′-TAC CHR AGG AGG AAG CCA C-3′ | Group I Clostridia |
| 12 | 5′-GTT CTT CCT AAT CTC TAC GCA T-3′ | |
| 13 | 5′-ACG CTA CTT GAG GAG GA-3′ | Group II Clostridia |
| 14 | 5′-GAG CCG TAG CCT TTC ACT-3′ | |
| 15 | 5′-GAW GAA GTA TYT CGG TAT GT-3′ | Group III Clostridia |
| 16 | 5′-CTA CGC WCC CTT TAC AC-3′ | |
| 17 | 5′-CTC AAC TTG GGT GCT GCA TTT-3′ | Clostridia (all groups) |
| 18 | 5′-ATT GTA GTA CGT GTG TAG CCC-3′ | |
| 19 | 5′-TTC CGG TTG ATC CYG CCG GA-3′ | Archaea |
| 20 | 5′-YCC GGC GTT GAM TCC AAT T-3′ | |
| 21 | 5′-TCY GKT TGA TCC YGS CRG AG-3′ | Archaea |
| 22 | 5′-TGG GTC TCG CTC GTT G-3′ | |
| 23 | 5′-ATT AGA TAC CCS BGT AGT CC-3′ | Archaea |
| 24 | 5′-GCC ATG CAC CWC CTC T-3′ | |
| 25 | 5′-GYG CAS CAG KCG MGA AW-3′ | Archaea |
| 26 | 5′-GGA CTA CVS GGG TAT CTA AT-3′ | |
| 29 | 5′-TGG TTG ATC CTG CCA GAG G-3′ | All Methanogens |
| 30 | 5′-CGG CTG GCA CCG GTC TTG C-3′ | |
| 31 | 5′-GGT GGT GTM GGA TTC ACA CAR TAY GCW ACA GC-3′ | Methanogen-specific directed |
| 32 | 5′-TTC ATT GCR TAG TTW GGR TAG TT-3′ | to the mcr gene encoding the |
| | | methyl coenzyme M reductase |
| 33 | 5′-GCM ATG CAR ATH GGW ATG TC-3′ | Methanogen-directed to |
| 34 | 5′-TCA TKG CRT AGT TDG GRT AGT-3′ | methyl coenzyme M reductase |
| 35 | 5′-TAA GGG CTG GGC CAA GT-3′ | Methanococcales |
| 36 | 5′-CAC CTA GTY CGC ARA GTT TA-3′ | |
| 37 | 5′-CGW AGG GAA GCT GTT AAG T-3′ | Methanobacterium |
| 38 | 5′-TAC CGT CGT CCA CTC CTT-3′ | |
| 39 | 5′-ATC GRT ACG GGT TGT GGG-3′ | Methanomicrobiales |
| 40 | 5′-CAC CTA ACG CRC ATH GTT TAC-3′ | |

DNA primers

| | | |
|---|---|---|
| 41 | 5′-GTA AAC GAT RYT CGC TAG GT-3′ | Methanosarcinales |
| 42 | 5′-GGT CCC CAC AGW GTA CC-3′ | |
| 43 | 5′-ATC AGT TTT CAC ATG GSG ATT GAT-3′ | Clostridium ragsdalei |
| 44 | 5′-GGG TTA AGC CCG GGT A-3′ | |
| 45 | 5′-TTA GTT TTT CAC ATG AAA TAC TAA-3′ | Clostridium carboxydivorans |
| 46 | 5′-AAG TTA AGC TCG GGA T-3′ | |

*wherein I is inosine; R is A or G; Y is C or T; K is G or T; M is A or C; S is G or C; W is A or T; B is G, C or T; D is G, A or T; H is A, C or T; V is G, C or A; and N is A, G, C or T. \hat{ }refers to 16s rDNA except when referencing Eukarya, in which case refers to the 17s or 18s rDNA gene.

In the assays shown in FIG. 4 and FIG. 5, amplification products were expected and found only from primer pairs which were specific for Eubacteria (1.5 kB product); Group I Clostridia (225 bp product); and all Clostridia (700 bp product). Archaeal primer pairs were expected and showed amplification products (900 bp) only in reactions containing pure archaeal DNA (lanes 8 and 9). These results demonstrate that the Eubacteria, all Clostridia and Group I Clostridia primers are all able to detect Group I Clostridia (Clostridium carboxidovorans cells or Clostridium ragsdalei cells). Additionally, archaeal primers did not produce amplification products against Eubacterial cells, but did show amplification products in reactions containing methanogen DNA. Thus, Eubacteria and Archaeabacteria may be readily detected in mixed cultures, such as from a contaminated fermentation process.

Verification of 16S-Based Ribosomal DNA Primers for PCR with Clostridial Subspecies

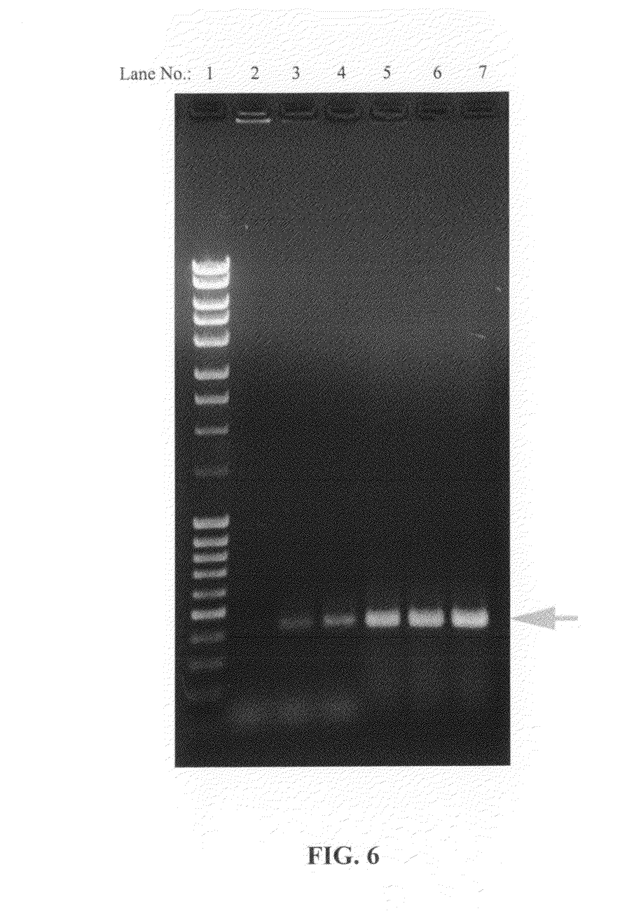

An rDNA PCR amplification reaction was carried out using a set of PCR primers specific for Clostridium carboxidovorans cells or Clostridium ragsdalei cells. FIG. 6 is an ethidium bromide stained 1% agarose gel showing the electrophoretic profile of rDNA amplification products from pure cultures of Clostridia. Reactions contained: Clostridium carboxidovorans cells and 16s ribosomal RNA domain-specific amplification primers specific for C carboxidovorans, SEQ ID NOs. 43, 44 (lanes 2, 3, 4 are three different annealing temperatures, 46° C., 50° C. and 58° C., respectively); Clostridium ragsdalei cells and 16s ribosomal RNA domain-specific amplification primers specific for C ragsdalei, SEQ ID NOs. 45, 46 (lanes 5, 6, 7 are three different annealing temperatures, 46° C., 50° C. and 58° C., respectively). Lane 1 is a 1 Kb mass ladder and shows that the amplification product is of the expected size (420 bp).

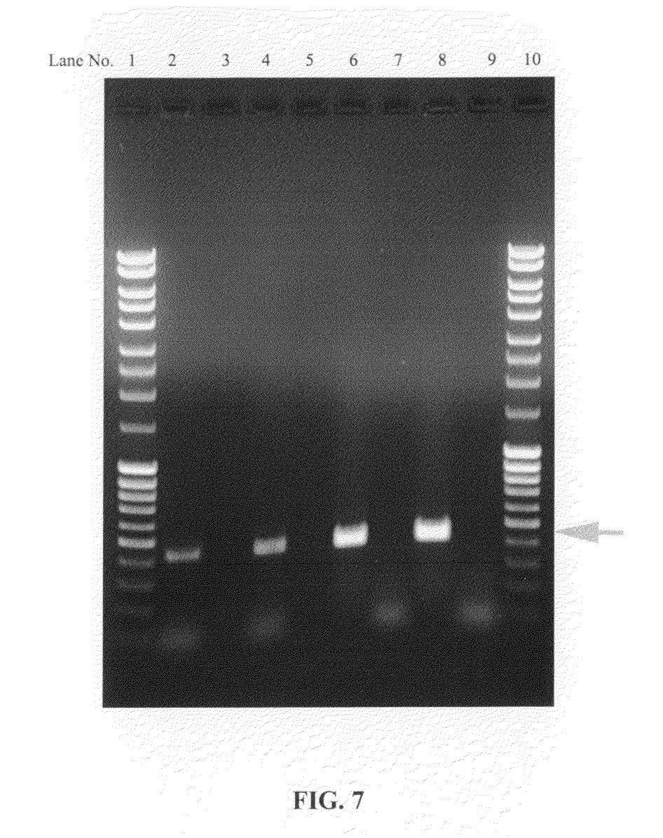

FIG. 7 shows an electrophoretic profile of rDNA amplification products from pure cultures of Clostridia using clostridia-based 16s ribosomal RNA domain-specific amplification primers on an ethidium bromide stained 1% agarose gel. Reactions contained: Clostridium carboxidovorans cells and C carboxidovorans specific primers, SEQ ID NOs. 43, 44 (lanes 2, 4 at annealing temperatures 46° C. and 50° C., respectively); Clostridium carboxidovorans cells and C ragsdalei specific primers, SEQ ID NOs. 45, 46 (lanes 3, 5 at annealing temperatures 46° C. and 50° C., respectively); Clostridium ragsdalei cells and C ragsdalei specific primers, SEQ ID NOs. 45, 46 (lanes 6, 8 at annealing temperatures 50° C. and 58° C., respectively); Clostridium ragsdalei cells and C carboxidovorans specific primers, SEQ ID NOs. 43, 44 (lanes 7, 9 at annealing temperatures 50° C. and 58° C., respectively). Lanes 1 and 10 are a 1 Kb mass ladder and show that the amplification product is of the expected size (420 bp).

Results from these two experiments demonstrate that primers directed to subspecies of Clostridia, such as the Group I Clostridium carboxidovorans or Clostridium ragsdalei, are able to hybridize with and produce amplification products only for their expected or suspected target. The results shown in FIG. 7 demonstrate that primers directed to Clostridium carboxidovorans did not cross react with Clostridium ragsdalei, and vice versa. Thus, specific subspecies may be readily detected in mixed cultures, such as from a contaminated fermentation process.

1. A method for determining the presence of a contaminating organism in a syngas fermentation process, the method comprising:
initiating the fermentation process using at least one syngas input;
obtaining a first sample from the fermentation process;
subjecting said first sample to a polymerase chain reaction using at least one set of oligonucleotide primers capable of hybridizing to a target sequence of a genomic nucleic acid from a suspected contaminating organism;
producing a first amplified product based on the polymerase chain reaction;
separating the first amplified product based on size; and
determining the presence of the suspected contaminating organism based on the first separated amplified product.

2. The method of claim 1, further comprising:
obtaining a second sample from the fermentation process;
subjecting said second sample to a polymerase chain reaction using at least one set of oligonucleotide primers capable of hybridizing to a target sequence of a genomic nucleic acid from a suspected contaminating organism;
producing a second amplified product based on the polymerase chain reaction;
separating the second amplified product based on size;
comparing the first separated amplified product and second separated amplified product; and
determining a change in contamination of the fermentation process based on the comparison.

3. The method of claim 1, further comprising:
obtaining a control sample including a known concentration of at least a first contaminating agent;
subjecting said control sample to a polymerase chain reaction using at least one set of oligonucleotide primers capable of hybridizing to a target sequence of said control sample;
producing a control amplified product based on the polymerase chain reaction;
separating the control amplified product based on size; and
comparing the first separated amplified product and the control amplified product, wherein the determination of the presence of the suspected contaminating organism is additionally based on the comparison.

4. The method of claim 1, wherein said contaminating organism is a unicellular microorganism selected from the group consisting of archaea, bacteria and phage.

5. The method of claim 4, wherein said contaminating organism is selected from the group consisting of Clostridia and Methanogen.

6. The method of claim 1, wherein said sample is selected from the group consisting of glycerol stocks, lyophilized cultures, agar stabs, agar plated cultures, seed cultures, effluent cultures and biofilm cultures.

7. The method of claim 1, wherein said sample is a fermentation aliquot selected at a time point from the fermentation process.

8. The method of claim 1, wherein separating the control amplified product based on size comprises:
reacting said first amplified product with at least one restriction endonuclease to produce a set of shorter fragments of said first amplified product.

9. The method of claim 1, wherein said separation based on size is by gel electrophoresis.

10. The method of claim 1, wherein said target sequence of said genomic nucleic acid is a portion of a 16s ribosomal RNA or 16s ribosomal DNA.

11. The method of claim 1, further comprising:
subjecting said control sample to a polymerase chain reaction using at least one additional set of oligonucleotide primers capable of hybridizing to a target sequence of said control sample, wherein each additional set of oligonucleotide primers being specific for a target sequence from at least an additional suspected contaminating organism.

12. The method of claim 1, wherein said oligonucleotide primers are selected from the group consisting of:

| | |
|---|---|
| 5′-CCT ACG GGR BGC ASC AG-3′; | (SEQ ID No. 1) |
| 5′-GGA CTA CNN GGG TAT CTA AT-3′; | (SEQ ID No. 2) |
| 5′-CCT ACG GGR BGC ASC AG-3′; | (SEQ ID No. 3) |
| 5′-GGA CTA CYV GGG TAT CTA AT-3′; | (SEQ ID No. 4) |
| 5′-AGA GTT TGA TCC TGG CTC AG-3′; | (SEQ ID No. 5) |
| 5′-GGT TAC CTT GTT ACG ACT T-3′; | (SEQ ID No. 6) |
| 5′-CTA CGG GIG GCI GCA GT-3′; | (SEQ ID No. 7) |
| 5′-GGA CTA CCI GGG TIT CTA A-3′; | (SEQ ID No. 8) |
| 5′-AGA GTT TGA TCC TGG CTC AG-3′; | (SEQ ID No. 9) |
| 5′-ACG GCT ACC TTG TTA CGA CTT-3′; | (SEQ ID No. 10) |
| 5′-TAC CHR AGG AGG AAG CCA C-3′; | (SEQ ID No. 11) |
| 5′-GTT CTT CCT AAT CTC TAC GCA | (SEQ ID No. 12) |
| T-3′; | |
| 5′-ACG CTA CTT GAG GAG GA-3′; | (SEQ ID No. 13) |
| 5′-GAG CCG TAG CCT TTC ACT-3′; | (SEQ ID No. 14) |
| 5′-GAW GAA GTA TYT CGG TAT GT-3′; | (SEQ ID No. 15) |
| 5′-CTA CGC WCC CTT TAC AC-3′; | (SEQ ID No. 16) |
| 5′-CTC AAC TTG GGT GCT GCA TTT-3′; | (SEQ ID No. 17) |
| 5′-ATT GTA GTA CGT GTG TAG CCC-3′; | (SEQ ID No. 18) |
| 5′-TTC CGG TTG ATC CYG CCC GA-3′; | (SEQ ID No. 19) |
| 5′-YCC GGC GTT GAM TCC AAT T-3′; | (SEQ ID No. 20) |
| 5′-TCY GKT TGA TCC YGS CRG AG-3′; | (SEQ ID No. 21) |
| 5′-TGG GTC TCG CTC GTT G-3′; | (SEQ ID No. 22) |
| 5′-ATT AGA TAC CCS BGT AGT CC-3′; | (SEQ ID No. 23) |
| 5′-GCC ATG CAC CWC CTC T-3′; | (SEQ ID No. 24) |
| 5′-GYG CAS CAG KCG MGA AW-3′; | (SEQ ID No. 25) |
| 5′-GGA CTA CVS GGG TAT CTA AT-3′; | (SEQ ID No. 26) |
| 5′-TGG TTG ATC CTG CCA GAG G-3′; | (SEQ ID No. 29) |
| 5′-CGG CTG GCA CCG GTC TTG C-3′; | (SEQ ID No. 30) |
| 5′-GGT GGT GTM GGA TTC ACA CAR TAY | (SEQ ID No. 31) |
| GCW ACA GC-3′; | |
| 5′-TTC ATT GCR TAG TTW GGR TAG | (SEQ ID No. 32) |
| TT-3′; | |
| 5′-GCM ATG CAR ATH GGW ATG TC-3′; | (SEQ ID No. 33) |
| 5′-TCA TKG CRT AGT TDG GRT AGT-3′; | (SEQ ID No. 34) |
| 5′-TAA GGG CTG GGC CAA GT-3′; | (SEQ ID No. 35) |
| 5′-CAC CTA GTY CGC ARA GTT TA-3′; | (SEQ ID No. 36) |
| 5′-CGW AGG GAA GCT GTT AAG T-3′; | (SEQ ID No. 37) |
| 5′- TAC CGT CGT CCA CTC CTT-3′; | (SEQ ID No. 38) |
| 5′- ATC GRT ACG GGT TGT GGG-3′; | (SEQ ID No. 39) |
| 5′-CAC CTA ACG CRC ATH GTT TAC-3′; | (SEQ ID No. 40) |
| 5′-GTA AAC GAT RYT CGC TAG GT-3′; | (SEQ ID No. 41) |
| 5′-GGT CCC CAC AGW GTA CC-3′; | (SEQ ID No. 42) |
| 5′-ATC AGT TTT CAC ATG GSG ATT | (SEQ ID No. 43) |
| GAT-3′; | |
| 5′-GGG TTA AGC CCG GGT A-3′; | (SEQ ID No. 44) |
| 5′-TTA GTT TTT CAC ATG AAA TAC | (SEQ ID No. 45) |
| TAA-3′; | |
| 5′-AAG TTA AGC TCG GGA T-3′; | (SEQ ID No. 46) |
| 5′-GTA GTC ATA TGC TTG TCT C-3′; | (SEQ ID No. 47) |
| 5′-TCC GCA GGT TCA CCT ACG GA-3′; | (SEQ ID No. 48) |
| 5′-GCA AGT CTG GTG CCA GCA GCC-3′; | (SEQ ID No. 49) |
| and | |
| 5′-CTT CCG TCA ATT CCT TTA AG-3′; | (SEQ ID No. 50) |

wherein I is inosine; R is A or G; Y is C or T; K is G or T; M is A or C; S is G or C; W is A or T; B is G, C or T; D is G, A or T; H is A, C or T; V is G, C or A; and N is A, G, C or T.

13. The method of claim 1, wherein obtaining a first sample from the fermentation process comprises:
subjecting said first sample to at least one sample preparation step;
adding a known amount of at least one known nucleic acid molecule as an internal standard, wherein said at least one known nucleic acid molecule is capable of hybridizing with the same set of oligonucleotide primers used to amplify said target sequence of a genomic nucleic acid from the suspected contaminating organism; and wherein separating the control amplified product based on size comprises:
quantitating an amount of amplified internal standard product; and
quantitatively determining an amount of the first amplified product using the amount of amplified internal standard product.

14. A method for determining the presence of a contaminating organism in a syngas fermentation process, comprising:
initiating the fermentation process using at least one syngas input;
obtaining a first sample from the fermentation process;
subjecting said first sample to a molecular beacon assay using at least one oligonucleotide probe capable of hybridizing to a target sequence of a genomic nucleic acid from a suspected contaminating organism to produce a visible signal;
quantifying said first visible signal; and
determining the presence of the suspected contaminating organism based on said first visible signal.

15. The method of claim 14, further comprising:
obtaining a control sample including a known concentration of at least a first contaminating agent;
subjecting said control sample to a molecular beacon assay using at least one oligonucleotide probe capable of hybridizing to a target sequence of a genomic nucleic acid from said control sample to produce a control visible signal; and
quantifying said control visible signal; and comparing the first visible signal and said control visible signal, wherein the determination of the presence of the suspected contaminating organism is additionally based on the comparison.

16. An oligonucleotide primer for use in a PCR assay for determining the presence, absence, or amount of a contaminating organism in a fermentation process, selected from the group consisting of:

| | |
|---|---|
| 5′-ATC AGT TTT CAC ATG GSG ATT | (SEQ ID No. 43) |
| GAT-3′; | |
| 5′-GGG TTA AGC CCG GGT A-3′; | (SEQ ID No. 44) |
| 5′-TTA GTT TTT CAC ATG AAA TAC | (SEQ ID No. 45) |
| TAA-3′; | |
| and | |
| 5′-AAG TTA AGC TCG GGA T-3′ | (SEQ ID No. 46) |

wherein S is G or C.

17. A kit for detecting the presence, absence, or amount of a contaminating organism in a syngas fermentation to produce liquid products, comprising:
at least one set of oligonucleotide primers, wherein said primers are capable of hybridizing to a target sequence of a genomic nucleic acid of a suspected contaminating organism to produce an amplified product in a polymerase chain reaction;
a control sequence; and
a set of control oligonucleotide primers, wherein said control primers are capable of hybridizing to the control sequence to produce an amplified product of a known length in a polymerase chain reaction.

18. The kit of claim 17, wherein said oligonucleotide primers are selected from the group consisting of:

| | |
|---|---|
| 5′-CCT ACG GGR BGC ASC AG-3′; | (SEQ ID No. 1) |
| 5′-GGA CTA CNN GGG TAT CTA AT-3′; | (SEQ ID No. 2) |
| 5′-CCT ACG GGR BGC ASC AG-3′; | (SEQ ID No. 3) |
| 5′-GGA CTA CYV GGG TAT CTA AT-3′; | (SEQ ID No. 4) |
| 5′-AGA GTT TGA TCC TGG CTC AG-3′; | (SEQ ID No. 5) |
| 5′-GGT TAC CTT GTT ACG ACT T-3′; | (SEQ ID No. 6) |
| 5′-CTA CGG GIG GCI GCA GT-3′; | (SEQ ID No. 7) |
| 5′-GGA CTA CCI GGG TIT CTA A-3′; | (SEQ ID No. 8) |
| 5′-AGA GTT TGA TCC TGG CTC AG-3′; | (SEQ ID No. 9) |
| 5′-ACG GCT ACC TTG TTA CGA CTT-3′; | (SEQ ID No. 10) |
| 5′-TAC CHR AGG AGG AAG CCA C-3′; | (SEQ ID No. 11) |
| 5′-GTT CTT CCT AAT CTC TAC GCA | (SEQ ID No. 12) |
| T-3′; | |
| 5′-ACG CTA CTT GAG GAG GA-3′; | (SEQ ID No. 13) |
| 5′-GAG CCG TAG CCT TTC ACT-3′; | (SEQ ID No. 14) |
| 5′-GAW GAA GTA TYT CGG TAT GT-3′; | (SEQ ID No. 15) |
| 5′-CTA CGC WCC CTT TAC AC-3′; | (SEQ ID No. 16) |
| 5′-CTC AAC TTG GGT GCT GCA TTT-3′; | (SEQ ID No. 17) |
| 5′-ATT GTA GTA CGT GTG TAG CCC-3′; | (SEQ ID No. 18) |
| 5′-TTC CGG TTG ATC CYG CCG GA-3′; | (SEQ ID No. 19) |
| 5′-YCC GGC GTT GAM TCC AAT T-3′; | (SEQ ID No. 20) |
| 5′-TCY GKT TGA TCC YGS CRG AG-3′; | (SEQ ID No. 21) |
| 5′-TGG GTC TCG CTC GTT G-3′; | (SEQ ID No. 22) |
| 5′-ATT AGA TAC CCS BGT AGT CC-3′; | (SEQ ID No. 23) |
| 5′-GCC ATG CAC CWC CTC T-3′; | (SEQ ID No. 24) |
| 5′-GYG CAS CAG KCG MGA AW-3′; | (SEQ ID No. 25) |
| 5′-GGA CTA CVS GGG TAT CTA AT-3′; | (SEQ ID No. 26) |
| 5′-TGG TTG ATC CTG CCA GAG G-3′; | (SEQ ID No. 29) |
| 5′-CGG CTG GCA CCG GTC TTG C-3′; | (SEQ ID No. 30) |
| 5′-GGT GGT GTM GGA TTC ACA CAR TAY | (SEQ ID No. 31) |
| GCW ACA GC-3′; | |
| 5′-TTC ATT GCR TAG TTW GGR TAG | (SEQ ID No. 32) |
| TT-3′; | |
| 5′-GCM ATG CAR ATH GGW ATG TC-3′; | (SEQ ID No. 33) |
| 5′-TCA TKG CRT AGT TDG GRT AGT-3′; | (SEQ ID No. 34) |
| 5′-TAA GGG CTG GGC CAA GT-3′; | (SEQ ID No. 35) |
| 5′-CAC CTA GTY CGC ARA GTT TA-3′; | (SEQ ID No. 36) |
| 5′-CGW AGG GAA GCT GTT AAG T-3′; | (SEQ ID No. 37) |
| 5′- TAC CGT CGT CCA CTC CTT-3′; | (SEQ ID No. 38) |
| 5′- ATC GRT ACG GGT TGT GGG-3′; | (SEQ ID No. 39) |
| 5′-CAC CTA ACG CRC ATH GTT TAC-3′; | (SEQ ID No. 40) |
| 5′-GTA AAC GAT RYT CGC TAG GT-3′; | (SEQ ID No. 41) |
| 5′-GGT CCC CAC AGW GTA CC-3′; | (SEQ ID No. 42) |
| 5′-ATC AGT TTT CAC ATG GSG ATT | (SEQ ID No. 43) |
| GAT-3′; | |
| 5′-GGG TTA AGC CCG GGT A-3′; | (SEQ ID No. 44) |
| 5′-TTA GTT TTT CAC ATG AAA TAC | (SEQ ID No. 45) |
| TAA-3′; | |
| 5′-AAG TTA AGC TCG GGA T-3′; | (SEQ ID No. 46) |
| 5′-GTA GTC ATA TGC TTG TCT C-3′; | (SEQ ID No. 47) |
| 5′-TCC GCA GGT TCA CCT ACG GA-3′; | (SEQ ID No. 48) |
| 5′-GCA AGT CTG GTG CCA GCA GCC-3′; | (SEQ ID No. 49) |
| and | |
| 5′-CTT CCG TCA ATT CCT TTA AG-3′; | (SEQ ID No. 50) |

wherein I is inosine; R is A or G; Y is C or T;
K is G or T; M is A or C; S is G or C; W is A or
T; B is G, C or T; D is G, A or T; H is A, C or
T; V is G, C or A; and N is A, G, C, or T.

19. The kit of claim 17, wherein said target sequence of said genomic nucleic acid is a portion of a 16s ribosomal RNA or 16s ribosomal DNA.

20. A kit for detecting the presence, absence, or amount of a contaminating organism in a syngas fermentation to produce liquid products, comprising:
at least one oligonucleotide probe, wherein said probe is capable of hybridizing to a target sequence of a genomic nucleic acid of a suspected contaminating organism to produce a visible signal;
a control sequence; and
a control oligonucleotide probe, wherein said control probe is capable of hybridizing to the control sequence to produce a visible signal.

21. The kit of claim 20, wherein said oligonucleotide probe and control oligonucleotide probe are molecular beacons.
